(19) United States
(12) Patent Application Publication
(10) Pub. No.: US 2026/0294381 A1
(43) Pub. Date: Oct. 1, 2026

(54) LIVER CAPSULE DEPTH AND ANGLE DETECTION

(71) Applicant: KONINKLIJKE PHILIPS N.V., EINDHOVEN (NL)

(72) Inventors: Man Minh Nguyen, Melrose, MA (US); Elizabeth Herbst, Cambridge, MA (US); Hua Xie, Cambridge, MA (US)

(21) Appl. No.: 19/474,782

(22) PCT Filed: Apr. 3, 2024
(86) PCT No.: PCT/EP2024/058973

§ 371 Date: Oct. 13, 2025

Related U.S. Application Data
(60) Provisional application No. 63/458,706, filed on Apr. 12, 2023.

(51) Int. Cl. A61B 8/08; A61B 8/00; G06T 7/00; G06T 7/73

(52) U.S. Cl. CPC A61B 8/085; A61B 8/4483; A61B 8/461; A61B 8/469; A61B 8/5223; A61B 8/5269; A61B 8/54; G06T 7/0012; G06T 7/73; G06T 2200/24; G06T 2207/10132; G06T 2207/30056

(57) ABSTRACT

An ultrasound system (100) includes a memory (151) that stores instructions; and a processor (152) that executes the instructions. When executed by the processor (152), the instructions cause the ultrasound system (100) to: obtain ultrasound images from a transducer array (113) of an ultrasound probe (110); detect, based on the ultrasound images, a depth of a liver capsule relative to the transducer array (113) and an angle of the transducer array (113) relative to the liver capsule; and generate feedback based on the depth and the angle.

### BACKGROUND

Detecting liver capsules is useful in liver disease diagnosis. For a normal liver, the liver capsule appears in ultrasound images as a linear structure with uniform thickness. For a diseased liver, the liver capsule appears in ultrasound images with uneven/wavy and sometimes broken outlines.

In ultrasound imaging, liver capsules also introduce undesirable acoustic effects with reverberation and aberration. The reverberation is due to ultrasound signals propagating in the tissue between the liver capsule and the transducer surface and then bouncing back and forth within the liver capsule. The aberration is due to the speed of sound varying in different mediums. The speed of sound in liver capsules and abdominal layers with a mix of fat and muscle tissue is different than the speed of sound in the liver. These artefacts lead to image quality degradation and inaccurate estimation of certain acoustic parameters used for liver disease assessment. For example, in attenuation imaging, the liver capsule causes estimation errors of attenuation coefficients in the region affected by reverberation, typically within regions up to twice the depth of the liver capsule. It is important to minimize the impact of these erroneous regions on the quantitative measurement used for diagnosis. An imaging region of interest should be placed below the depth of the liver capsule (preferably twice the liver capsule depth in the case of attenuation imaging), but there is currently no mechanism to determine the depth of the liver capsule.

Separately, elastography requires transmitting long acoustic push pulses which generate shear waves propagating inside tissue. These waves are then tracked for calculating velocity (m/s), which is subsequently converted to tissue stiffness in terms of shear or young modulus (kPa). The layer patterns of liver capsules and the abdominal tissues potentially change the way shear waves are generated and therefore affect the tracked velocity of these shear waves. One guideline for shear wave elastography recommends following three parallel lines, where the liver capsule is parallel to the transducer surface.

### SUMMARY

According to an aspect of the present disclosure, an ultrasound system includes a memory that stores instructions; and a processor that executes the instructions. When executed by the processor, the instructions cause the ultrasound system to: obtain ultrasound images from a transducer array of an ultrasound probe; detect, based on the ultrasound images, a depth of a liver capsule relative to the transducer array and an angle of the transducer array relative to the liver capsule; and generate feedback based on the depth and the angle.

According to another aspect of the present disclosure, an ultrasound probe includes a transducer array; a memory that stores instructions; and a processor that executes the instructions. When executed by the processor, the instructions cause the ultrasound probe to obtain ultrasound images from the transducer array; detect, based on the ultrasound images, a depth of a liver capsule relative to the transducer array and an angle of the transducer array relative to the liver capsule; and generate feedback based on the depth and the angle.

According to another aspect of the present disclosure, an ultrasound probe comprises a transducer array, a memory that stores instructions, and a processor that executes the instructions. A method of operating the ultrasound probe includes, when the instructions are executed by the processor, obtaining ultrasound images from the transducer array; detecting, based on the ultrasound images, a depth of a liver capsule relative to the transducer array and an angle of the transducer array relative to the liver capsule; and generating feedback based on the depth and the angle.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for liver capsule depth and angle detection, in accordance with a representative embodiment.

FIG. 2 illustrates an ultrasound probe for liver capsule depth and angle detection, in accordance with a representative embodiment.

FIG. 3 illustrates attenuation imaging artefacts due to a liver capsule and abdominal depth in liver capsule depth and angle detection, in accordance with a representative embodiment.

FIG. 4 illustrates positioning of a liver capsule and field of view (FOV) box in liver capsule depth and angle detection, in accordance with a representative embodiment.

FIG. 5 illustrates a progression of an algorithm with one option of user feedback for liver capsule depth and angle detection, in accordance with a representative embodiment.

FIG. 6 illustrates a progression of an algorithm with one option of user feedback for liver capsule depth and angle detection, in accordance with a representative embodiment.

FIG. 7 illustrates a dedicated button in a user interface for liver capsule depth and angle detection, in accordance with a representative embodiment.

FIG. 8 illustrates suggested annotations in a user interface for liver capsule depth and angle detection, in accordance with a representative embodiment.

FIG. 9 illustrates a method for liver capsule depth and angle detection, in accordance with a representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, feedback may be generated based on detecting the depth of a liver capsule relative to a transducer array and an angle of the transducer relative to the liver capsule. Detection of the depth may be used to ensure placement of the region of interest properly by the sonographer. Detection of the angle may be used to prompt the sonographer to improve parallelism between the liver capsule and the transducer array, insofar as the transducer array is ideally parallel to the liver capsule for elastography imaging. The design of layered estimated models which improve the accuracy of sound speed estimation may benefit from detecting liver capsules when the depth and angle are fed back in real-time in this manner. Liver B-mode image quality may also be improved using corrected sound speeds from more accurate speed-of-sound estimation accounting for liver capsule segmentation.

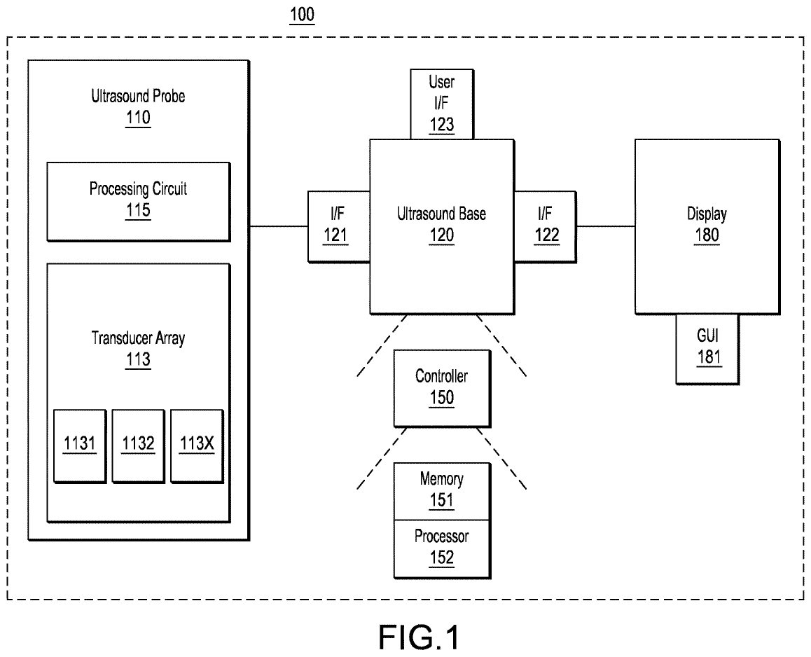

FIG. 1 illustrates a system 100 for liver capsule depth and angle detection, in accordance with a representative embodiment.

The system 100 in FIG. 1 is a system for liver capsule depth and angle detection and includes components that may be provided together or that may be distributed. The system 100 includes an ultrasound probe 110, an ultrasound base 120 and a display 180. The ultrasound probe 110 includes a transducer array 113 and a processing circuit 115. The transducer array 113 includes individual transducer elements including a first transducer element 1131, a second transducer element 1132 and an xth transducer element 113x. The ultrasound base includes a first interface 121, a second interface 122, a user interface 123 and a controller 150. The controller 150 includes a memory 151 that stores instructions and a processor 152 that executes the instructions. The instructions stored in the memory 151 may include parameters and/or commands for operations implemented by or at least using the ultrasound probe 110. In some embodiments, multiple different elements of the system 100 in FIG. 1 may include a controller such as the controller 150. For example, the processing circuit 115 of the ultrasound probe 110 may comprise a controller separate from the controller 150. The display 180 includes a graphical user interface 181. One or more of the interfaces of the ultrasound base 120 and the display 180 may include ports, disk drives, wireless antennas, or other types of receiver circuitry that connect to other electronic elements. One or more of the interfaces may also include user interfaces such as buttons, keys, a mouse, a microphone, a speaker, or other elements that users can use to interact with such as to enter instructions and receive output.

In FIG. 1, the ultrasound probe 110 may be connected wirelessly or by wire with the ultrasound base 120 via the first interface 121. For example, the ultrasound probe 110 may be connected to the ultrasound base 120 by a cord. The display 180 may be connected wirelessly or by wire with the ultrasound base via the second interface 122.

The ultrasound probe 110 includes the transducer array 113, and the transducer array 113 converts electrical energy into sound waves which bounce off of body tissue, and receives echoes of the sound waves and converts the echoes into electrical energy. The transducer array 113 may include dozens, hundreds or thousands of individual transducer elements. The ultrasound probe 110 may transmit a beam to produce images and may detect the echoes. The processing circuit 115 may control transmission of beams from the transducer array 113, and may be used to process ultrasound images captured by the transducer array 113 of the ultrasound probe 110. For example, the processing circuit 115 may include one or more beamformer used to form beams to be transmitted from the transducer array 113, and may include a transmit controller and/or sensors used to control, sense and record orientations of beams with respect to the ultrasound probe 110.

The ultrasound base 120 may be or otherwise include an ultrasound cart. In addition to the controller 150, the ultrasound base 120 may include buttons as or other interactive mediums as the user interface 123. The user interface 123 may comprise multiple user interfaces such as buttons, and the buttons may correspond to different functions of the ultrasound base 120. Sonographers may be well versed to recognize which buttons to press to control different functions of the ultrasound base 120 and the ultrasound probe 110. The user interface 123 may also be or include lights and/or annunciators to show liver capsule detection and correct angle, though such a user interface may also or alternatively be provided on the ultrasound probe 110.

Memory of a controller described herein may include one or more memories such as a main memory and/or a static memory, where such memories may include instructions executed by a processor and may communicate with each other and other elements of the controller via one or more buses. Memory is a tangible storage medium for storing data and/or executable software instructions, and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory used to store instructions for a controller may be used to implement some or all aspects of methods and processes described herein, along with data used in such methods and processes. The memory used to store instructions may be implemented by any number, type and combination of random access memory (RAM) and read-only memory (ROM), for example. In embodiments in which memories store various types of instructions and information, a processor may cause a controller in the processing circuit 115 and/or the controller 150 in the ultrasound base to perform various steps and methods using the instructions and information according to the present teachings. Furthermore, updates to the methods and processes described herein may also be stored in such a memory.

The various types ROM and RAM may include any number, type and combination of computer-readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. A computer readable storage medium is defined to be any medium that constitutes patentable subject matter under 35 U.S.C. § 101 and excludes any medium that does not constitute patentable subject matter under 35 U.S.C. § 101. Examples of such media include non-transitory media such as computer memory devices that store information in a format that is readable by a computer or data processing system. More specific examples of non-transitory media include computer disks and non-volatile memories.

The controller 150 and other controllers described herein are representative of one or more processing devices. In embodiments in which controllers comprise memories that store instructions and processors that execute the instructions, the controllers are configured to execute software instructions stored in such memories to perform functions as described in the various embodiments herein. The processor 152 and other processors and processing circuits described herein may be implemented by field programmable gate arrays (FPGAs), systems on a chip (SOC), a central processing unit, a computer processor, a microprocessor, a graphics processing unit (GPU), a microcontroller, a state machine, programmable logic device, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices. The term “processor” as used herein encompasses an electronic component able to execute a program or machine executable instruction. References to a device comprising “a processor” should be interpreted to include more than one processor or processing core, as in a multi-core processor.

In the system 100, the display 180 may be used as a user interface to display the liver capsule in ultrasound images on the display 180, and mark the liver capsule in the ultrasound images displayed on the display. The display 180 may be used, for example, to mark liver capsule boundaries and otherwise display angles between the transducer array 113 and the liver capsule. The display may also be used to alert users when large angles exist between the transducer array 113 and the liver capsule. Guidelines for liver elastography imaging call for parallelism between the transducer array 113 and the liver capsule. The region of interest used as a target by sonographers may comprise a trapezoid. In some ultrasound systems, the guidelines call for sonographers to try and achieve three parallel lines showing parallelism between the transducer array 113, the liver capsule, and the nearest horizontal edge of the region of interest. Large angles may potentially affect quantification in shear wave elastography, so the display 180 may be used to help minimize concerns with the reliability of shear wave elastography quantification.

The display 180 may be local to the ultrasound base 120 or may be remotely connected to the ultrasound base 120. The display 180 may be connected to the ultrasound base 120 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may also include one or more input interface(s) such as those noted above that may connect to other elements or components, as well as an interactive touch screen configured to display prompts to users and collect touch input from users. The graphical user interface 181 may display any of the visual user interfaces described with respect to embodiments herein.

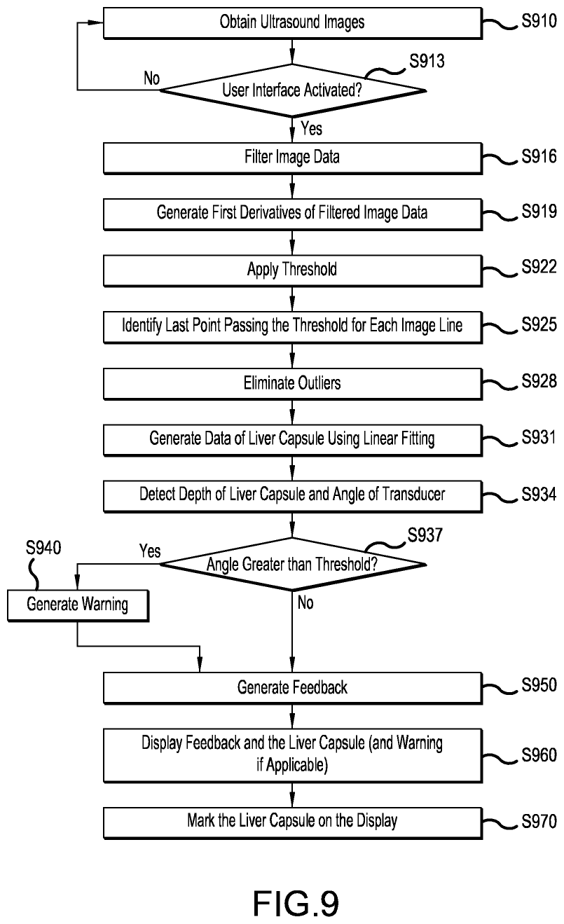

The controller 150 may implement a method as shown in and described with respect to FIG. 9. The controller 150 may perform some of the operations described herein directly and may implement other operations described herein indirectly. For example, the controller 150 may indirectly control operations such as by generating and transmitting content to be displayed on the display 180. The controller 150 may directly control other operations such as logical operations performed by the processor 152 executing instructions from the memory 151 based on input received from electronic elements and/or users via the interfaces. Accordingly, the processes implemented by the controller 150 when the processor 152 executes instructions from the memory 151 may include steps not directly performed by the controller 150.

As one example of operations performed by or using the controller 150, the system 100 includes the memory 151 that stores instructions; and a processor 152 that executes the instructions. When executed by the processor 152, the instructions cause the system 100 to: obtain ultrasound images from the transducer array 113 of the ultrasound probe 110; detect, based on the ultrasound images, a depth of a liver capsule relative to the transducer array 113 and an angle of the transducer array 113 relative to the liver capsule; and generate feedback based on the depth and the angle. The feedback may be output by the ultrasound probe 110 such as via haptic feedback, by the ultrasound base 120 such as by audible or visual feedback, and/or by the display 180 on the graphical user interface 181. The graphical user interface 181 may also be part of or separate from the display 180. For example, in some systems a touch screen may be provided separately from the display 180 for users to adjust settings. In other systems, the graphical user interface 181 is shown on the display 180.

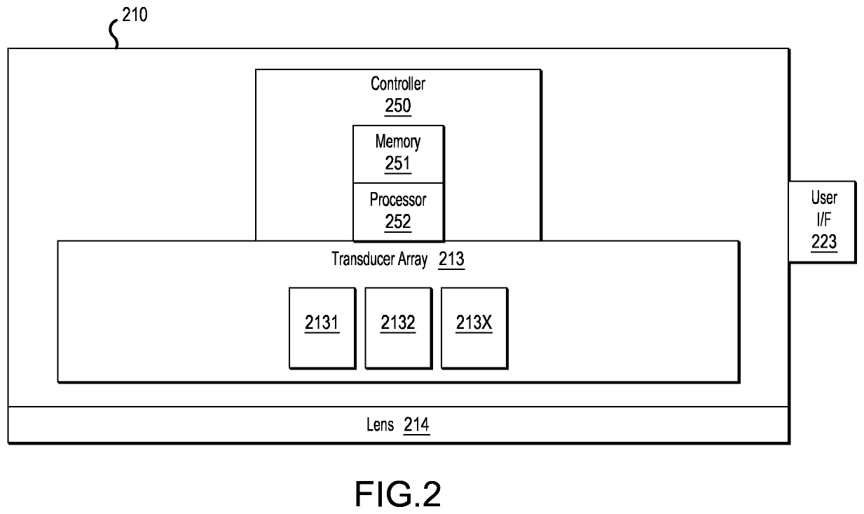

FIG. 2 illustrates an ultrasound probe for liver capsule depth and angle detection, in accordance with a representative embodiment.

The ultrasound probe 210 includes a transducer array 213, a lens 214, a user interface 223 and a controller 250. The controller 250 includes a memory 251 that stores instructions and a processor 252 that executes the instructions.

The transducer array 213 may correspond to the transducer array 113 and may comprise an array of individually controllable transducer elements. The transducer array 213 converts electrical energy into sound waves which bounce off of body tissue, and receives echoes of the sound waves and converts the echoes into electrical energy. The transducer array 213 may include dozens, hundreds or thousands of individual transducer elements. The ultrasound probe 210 may transmit a beam to produce images and may detect the echoes.

The lens 214 may be used to focus the beam transmitted by the ultrasound probe 210.

The user interface 223 may comprise one or more user interfaces, including a screen, a speaker, one or more buttons, or other types of user interfaces. A screen used as the user interface 223 may be used to mark liver capsule boundaries and angles, and alert users with large angles between the transducer array 213 and the liver capsule. Large angles may potentially affect shear wave elastography quantification, so the display 180 may be used to help minimize concerns with the reliability of shear wave elastography quantification. The user interface 223 may also be or include lights and/or annunciators to show liver capsule detection and correct angle. Although not shown in FIG. 2, the display 180 may be provided with the ultrasound probe 210 even when the ultrasound probe 210 is provided in a system without an ultrasound base such as the ultrasound base 120 in FIG. 1. Either the user interface 223 or a display such as the display 180 may be configured to display lights and/or annunciators to show liver capsule detection and correct angle.

The controller 250 may process ultrasound images captured by the transducer array 213 of the ultrasound probe 210. Alternatively, the ultrasound images captured by the transducer array 213 may be processed off of the ultrasound probe 210, such as by using an application on a separate mobile device which receives the ultrasound images from the ultrasound probe 210. The controller 250 may implement a method as shown in and described with respect to FIG. 9. The controller 250 may perform some of the operations described herein directly and may implement other operations described herein indirectly.

As one example of operations performed by or using the controller 250, the ultrasound probe 210 includes the memory 251 that stores instructions; and a processor 252 that executes the instructions. When executed by the processor 252, the instructions cause the ultrasound probe 210 to: obtain ultrasound images from the transducer array 213 of the ultrasound probe 210; detect, based on the ultrasound images, a depth of a liver capsule relative to the transducer array 213 and an angle of the transducer array 213 relative to the liver capsule; and generate feedback based on the depth and the angle. The feedback may be output by the ultrasound probe 210 such as via haptic feedback or visual feedback when the user interface 223 comprises a screen and/or audible feedback when the user interface 223 comprises a speaker. For example, the ultrasound base 120 of the system 100 or the ultrasound probe 210 may be configured to output the feedback for display on a display such as the display 180.

Although not shown in FIG. 2, the ultrasound probe 210 may send image data to a smartphone or tablet, and the image data may include the liver capsule identified in accordance with the teachings herein. In some embodiments, warnings to adjust the position of the ultrasound probe 210 may be provided via the smartphone or tablet.

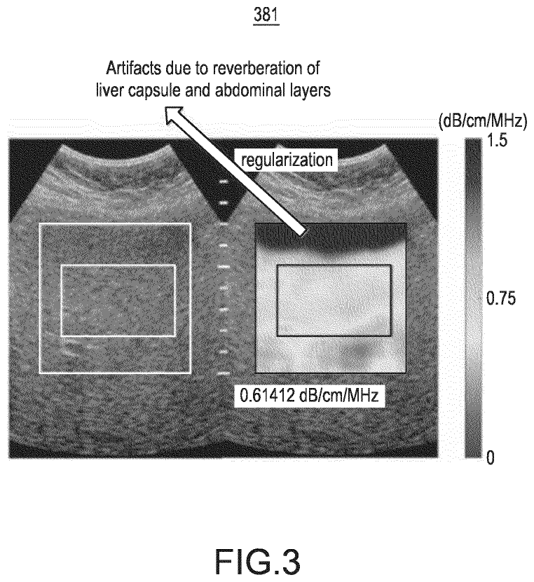

FIG. 3 illustrates attenuation imaging artefacts due to a liver capsule and abdominal depth in liver capsule depth and angle detection, in accordance with a representative embodiment.

In attenuation imaging, the liver capsule causes estimation errors of attenuation coefficients in the region affected by reverberation, typically within regions lower than twice the depth of the liver capsule. Attenuation results in artefacts as shown in FIG. 3 in the region from which the arrow emanates on the user interface 381. For example, the region from which the arrow emanates may be shown in red and this reflects artefacts due to reverberation of the liver capsule and abdominal areas. The reverberation is from the signal bouncing back and forth between the transducer surface and the liver capsule, and this results in artefacts in an attenuation imaging mode.

It is important to minimize the impact of these erroneous regions on the quantitative measurement used for diagnosis, and this is one important reason why a sonographer may be guided to adjust targeting of the imaging depth and/or the quantification region of the beam from the ultrasound probe 110 or the ultrasound probe 210, and/or to bring the transducer array parallel to the liver capsule. Guidance may be provided via a screen on the display 180, via a screen on the ultrasound probe 210, via haptic feedback from the ultrasound probe 110 or the ultrasound probe 210, or via audible feedback from a speaker. The sonographer may be guided so that the region of interest for quantification is not placed in the area where artefacts occur. A sonographer may be enabled to adjust the imaging depth and region of interest (ROI) using a control on the ultrasound base 140 such as the user interface 123, or otherwise on a user interface on the ultrasound probe 110. A user interface used to adjust the imaging depth and ROI may include touch screen, knobs and/or buttons.

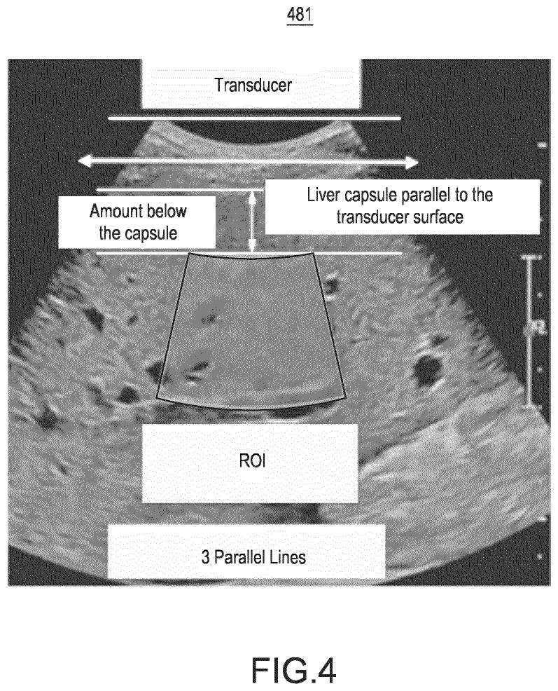

FIG. 4 illustrates positioning of a liver capsule and field of view (FOV) box in liver capsule depth and angle detection, in accordance with a representative embodiment.

Elastography requires transmitting long acoustic push pulses which generate shear waves propagating inside tissue. These waves are then tracked for calculating velocity (m/s), which is subsequently converted to tissue stiffness in terms of shear or young modulus (kPa). The layer patterns of liver capsules and the abdominal tissues potentially change the way shear waves are generated and therefore affect the tracked velocity of these shear waves. One guideline for shear wave elastography recommends following three parallel lines, where the liver capsule is parallel to the transducer surface. The three parallel lines are shown in FIG. 4.

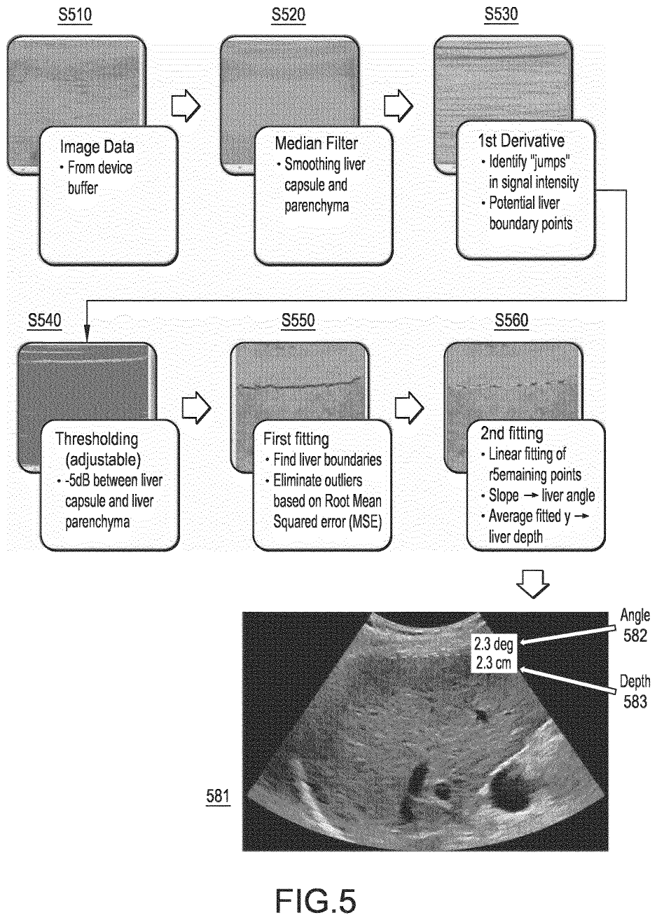

FIG. 5 illustrates a progression of an algorithm with one option of user feedback for liver capsule depth and angle detection, in accordance with a representative embodiment.

The progression in FIG. 5 reflect steps of an algorithm implemented by, for example, the controller 150 in FIG. 1 or the controller 250 in FIG. 2.

At S510, image data is obtained by an ultrasound probe such as the ultrasound probe 110 or the ultrasound probe 210. The image data may be buffered in a device buffer of the ultrasound probe, and then obtained from the device buffer.

At S520, the image data is median filtered to remove speckle variability in the liver capsule and parenchyma. The median filtering at S520 is a form of spatial filtering to remove speckle appearance and smooth the liver capsule and parenchyma. For example, the ultrasound base 120 of the system 100 or the ultrasound probe 210 may be configured to filter image data of the ultrasound images to remove speckle variability.

At S530, a first derivative of the median filtered image data is calculated and then applied to identify the changes in the signal intensities. The changes in signal intensities reflect the slope of the signal. In other words, big changes in signal changes can be identified from the first derivatives calculated at S530. The first derivative may identify jumps in signal intensity, along with potential liver boundary points. For example, the ultrasound base 120 of the system 100 or the ultrasound probe 210 may be configured to generate first derivatives of the filtered image data to identify changes in signal intensities relative to depth.

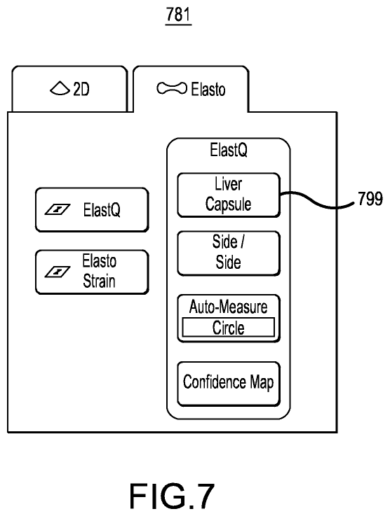

At S540, thresholding is performed by applying thresholds on the first derivatives to identify possible liver capsule boundaries. The thresholding at S540 may be based on an assumption that the brightness of the liver capsule and liver parenchyma is notably different. For example, the thresholding at S540 may be performed based on an assumption of a 5 decibel (5 dB) difference between the brightness of the liver capsule and the liver parenchyma. While the 5 decibel threshold is robust for identifying the liver capsule boundaries, in other embodiments the threshold may be adjusted such as for cases where the contrast between the liver capsule and parenchyma is not apparent. The ability to adjust the threshold may be provided along with the button 799 shown in and described with respect to FIG. 7 below. In some embodiments, instructions executed by the ultrasound base 120 of the system 100 or the ultrasound probe 210 may be configured to cause the system 100 or the ultrasound probe to adjust a threshold used to detect the liver capsule based on user input.

At S550, for each image line, the point representing the liver capsule is identified as the last point that passes the threshold inside a search window. This search window may be established based on the distributions of points that pass the threshold at S540. The first fitting at S550 may find liver boundary points and may eliminate outliers based, for example, on a root mean squared error (MSE). For example, the ultrasound base 120 of the system 100 or the ultrasound probe 210 may be configured to eliminate outliers of possible capsule boundary points. Outliers of the possible capsule boundary points may be eliminated after the first linear fitting Yfit=a1*X+b, where x and y are the lateral and depth of the points, respectively. The criteria for outlier determination may be the squared errors (y−yfit)2.

At S560, a second linear fitting is calculated. The second linear fitting may use an equation Y=a2*X+b2 and may be performed to draw the liver capsule. The angle of the liver capsule can be determined by slope a2 and the displayed depth can be the average of Y. The result of the second linear fitting may comprise an identification of the liver capsule, and the identification of the liver capsule enables feedback that can be provided to the sonographer. The relevant surface of the liver capsule drawn at S560 is the top surface between the liver tissue below and the muscle and fat above, though sides and a bottom of the liver capsule may also be identified and drawn in the method of FIG. 5.

The ultrasound image may be displayed on the user interface 581 after the second linear fitting, and may show an angle 582 of the liver capsule and a depth 583 calculated at S560. The feedback provided on the user interface 581 may be used by the sonographer to prompt adjustments of the position of the ultrasound probe. Additionally, the liver capsule identified in FIG. 5 may be shown in a b-mode ultrasound image, such as by superimposition.

Although embodiments herein are primarily described in the context of real-time feedback, in some embodiments an ultrasound session may be completed and most or all ultrasound images from the ultrasound session may be processed afterwards to identify ultrasound images in which the depth and angle are acceptable. For example, ultrasound images from an ultrasound session may be quantified after the ultrasound session, and the liver capsule may be drawn in images with a region of interest at a proper depth. Other ultrasound images from the ultrasound session may be discarded.

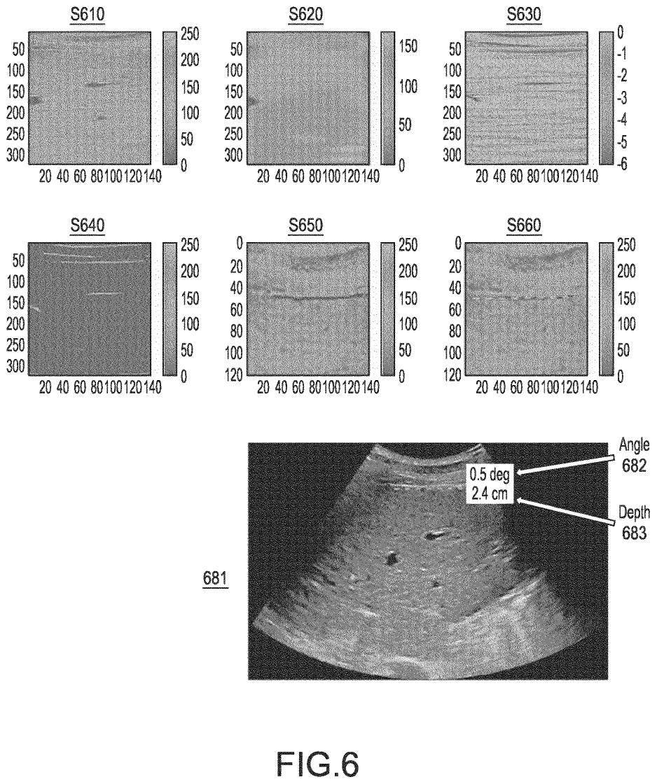

FIG. 6 illustrates a progression of an algorithm with one option of user feedback for liver capsule depth and angle detection, in accordance with a representative embodiment.

FIG. 6 illustrates another example of a progression similar to the progression in FIG. 5. In the example progression of FIG. 6, the contrast between the fat/muscle layer and parenchyma is less apparent. However, the robustness of the algorithm is illustrated in FIG. 6 insofar as the liver capsule is detected along with the angle 682 of the liver capsule and the depth 683.

The progression in FIG. 6 again reflects steps of an algorithm implemented by, for example, the controller 150 in FIG. 1 or the controller 250 in FIG. 2.

At S610, image data is obtained by an ultrasound probe such as the ultrasound probe 110 or the ultrasound probe 210. At S620, the image data is median filtered to remove speckle variability in the liver capsule and parenchyma. At S630, a first derivative of the median filtered image data is calculated and then applied to identify the changes in the signal intensities. At S640, thresholding is performed by applying thresholds on the first derivatives to identify possible liver capsule boundaries. At S650, for each image line, the point representing the liver capsule is identified as the last point that passes the threshold inside a search window. At S660, a second linear fitting is calculated. The details of the steps in FIG. 6 are similar or identical to those for the steps in FIG. 6, and are therefore not repeated. However, contrast between the fat/muscle layer and parenchyma is less apparent in FIG. 6 than in FIG. 5, though the angle 682 of the liver capsule and the depth 683 are still obtained as shown on the user interface 681.

The ultrasound image may be displayed on the user interface 681 after the second linear fitting, and may show an angle 682 of the liver capsule and a depth 683 calculated at S660. The feedback provided on the user interface 681 may be used by the sonographer to prompt adjustments of the position of the ultrasound probe.

FIG. 7 illustrates a dedicated button 799 in a user interface for liver capsule depth and angle detection, in accordance with a representative embodiment.

The button 799 may be added to a current user interface on the ultrasound base 120, the display 180 or the ultrasound probe 210 to turn on/off the liver capsule angle and depth detection feature described herein. The button 799 may be added to any ultrasound imaging mode where liver capsule artefacts (reverberation and aberration) can affect the quantitative measurements used for liver assessment.

In other embodiments, a knob switch may be used rather than the button 799 to enable a sonographer to select liver capsule angle and depth detection. In some embodiments, multiple user interface mechanisms may be provided to enable selection of liver capsule angle and depth detection.

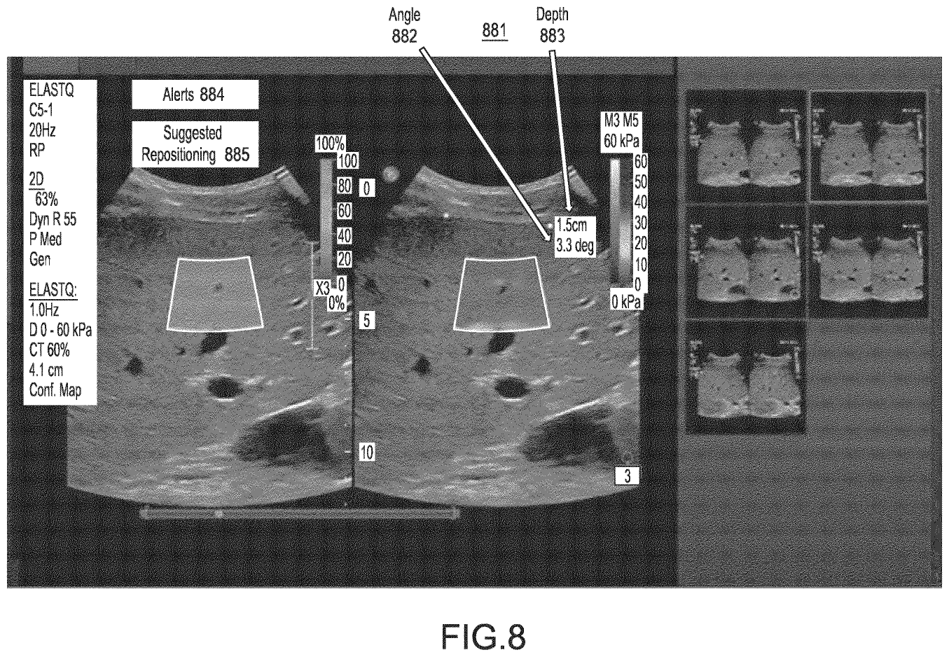

FIG. 8 illustrates suggested annotations in a user interface for liver capsule depth and angle detection, in accordance with a representative embodiment.

In the user interface 881, annotations are added for liver capsule depth 883 and angle 882. Feedback to users may also be displayed on the user interface 881. Examples of feedback include alerts 884 to the sonographer when the liver capsule angle is greater than a certain threshold such as degrees, and suggested repositioning 885 of region(s) of interest (ROI(s)) deeper to avoid artefacts due to the liver capsule.

FIG. 9 illustrates a method for liver capsule depth and angle detection, in accordance with a representative embodiment.

The method of FIG. 9 comprises an algorithm to detect liver capsule depth and angle through image data, and may be performed by the ultrasound base in FIG. 1 or the ultrasound probe 210 in FIG. 2. The method of FIG. 9 starts at S910 by obtaining ultrasound images. The ultrasound images may be obtained by the ultrasound probe 110 in FIG. 1 or the ultrasound probe 210 in FIG. 2. The image data may be initially buffered, and the retrieved from a device buffer for processing.

At S913, a determination is made as to whether a user interface has been activated. The user interface may be a dedicated soft button such as the button 799 in FIG. 7, or a dedicated hard button or another type of user interface which may be used to select a logical function in the controller 150 of the ultrasound base 120 in FIG. 1 or the controller 250 of the ultrasound probe 110 in FIG. 2.

If the user interface is not activated (S913=No), the method returns to S910. If the user interface is activated (S913=Yes), at S916 the image data is filtered. The filtering at S916 may be performed to remove speckle variability, and may include filtering by a median filter.

At S919 the method of FIG. 9 includes generating first derivatives of filtered image data. The first derivatives of the filtered image data may be generated to identify changes in signal intensities relative to depth.

At S922, a threshold is applied to the first derivates of the filtered image data. The threshold may be applied to identify possible liver capsule boundaries.

At S925, a last point passing the threshold is identified for each image line. The last point passing the threshold may be identified for each image line and may be identified from candidates inside a search window. The last points are identified as representing the liver capsule.

At S928, outliers are eliminated. The eliminated outliers are of possible capsule boundary points identified at S925. For example, outliers may be identified for elimination based on root mean squared error (MSE).

At S931, data of the liver capsule is generated using linear fitting.

At S934, a depth of a liver capsule and an angle of a transducer are detected. The detection at S934 is useful in liver disease diagnosis, insofar as the thickness of the fat in the liver capsule may be useful for monitoring for diseases related to the metabolism and/or cardiovascular system such as obesity, diabetes and heart diseases. For example, a measured thickness of the liver capsule may be compared to a threshold to determine whether the measurement of the thickness indicates a relatively-increased likelihood of the presence of a disease. An indication of disease likelihood may be displayed in real-time such as on the user interface 881 in FIG. 8, or later when a clinician is reviewing ultrasound readings taken at one time or at different times.

In some embodiments, longitudinal measurements of liver capsule thickness may be tracked based on the method of FIG. 9 being performed over time. The longitudinal measurements may be used for monitoring patient lifestyle change and treatment effectiveness, such as for obesity or metabolism disorders. An indication of patient lifestyle change and/or treatment effectiveness may be displayed in real-time such as on the user interface 881 in FIG. 8, or later when a clinician is reviewing ultrasound readings taken at one time or at different times.

At S937, a determination is made as to whether the angle is greater than a threshold, and if greater than the threshold (S937=Yes), a warning is generated at S940. The warning at S940 is used to improve and optimize the parallelism between the transducer array and the liver capsule, insofar as liver capsules introduce undesirable acoustic effects with reverberation and aberration in ultrasound imaging. The reverberation is based on ultrasound signals bouncing within the liver capsule, and the aberration is due to different speeds of sound in different mediums. The artefacts lead to image quality degradation and inaccurate estimation of certain acoustic parameters used for liver disease assessment, so the warning at S940 is used to improve or even optimize the parallelism.

If the angle is not greater than the threshold (S937=No), feedback is generated at S950. The feedback may be haptic, visual and/or audible, and may include a warning when the warning is generated at S940. Otherwise, the feedback generated at S950 may include readings of the angle and/or depth as described herein. Although not shown in FIG. 9, when the warning is generated at S940 the method of FIG. 9 may return to S910 during and/or after showing the warning from S940 as the feedback generated at S950.

At S960, the feedback and the liver capsule are displayed, along with a warning from S937 if applicable. For example, the feedback and the liver capsule may be displayed on the display 180 from FIG. 1. The liver capsule and feedback may be displayed in the ultrasound images on a display such as the display 180 in FIG. 1. For example, the feedback and the positions of the liver capsule may be superimposed on the ultrasound images.

At S970, the liver capsule is marked on the display. Having liver capsules identified and marked on images in S970 can help guide sonographers to position ultrasound probes and perform caliper placement for quantification, improving users' workflow and diagnosis accuracy. The display of the liver capsule at S970 may include a quantifiable assessment of liver capsule thickness which may be used for monitoring obesity, metabolism, risks of diabetes, and heart diseases. An indication of the quantifies assessment of liver capsule thickness and relationship with one or more diseases and conditions may be displayed in real-time such as on the user interface 881 in FIG. 8, or later when a clinician is reviewing ultrasound readings taken at one time or at different times.

Additionally, the liver capsule identified in FIG. 6 may be shown in a b-mode ultrasound image, such as by superimposition. Numbers on the side of the b-mode ultrasound image may indicate the pixel depth, and the pixel depth may be converted automatically into actual depth of the liver capsule in the b-mode ultrasound image.

The steps of the method of FIG. 9 do not particularly require raw ultrasound raw data. The method of FIG. 9 may operate on digital imaging and communication in medicine (DICOM) data, which makes it easy to deploy in live scanning mode as well as in review or offline modes. The steps herein can be used in different imaging modes including elastography, attenuation imaging, and speed-of-sound imaging.

Additionally, the method of FIG. 9 allows sonographers to assess the current view and adjust as needed based on the warning generated at S937 and/or the feedback generated at S950. The ability to improve parallelism allows for a more accurate quantitative assessment of liver biomarkers including more accurate elastography and more accurate estimations of the speed of sound.

While the teachings herein may assume use of frequencies used regularly for quantitative modes, high-frequency imaging may be used for broadband transducers specifically for improving liver capsule detection while maintaining the regular frequencies for the quantitative modes. The high-frequency imaging may provide improved spatial resolution at the expense of penetration. The high frequencies may be achieved with the same transmit events as the quantitative modes through bandpass filters.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, liver capsule depth and angle detection enables generation of feedback based on detecting the depth of a liver capsule relative to a transducer array and an angle of the transducer relative to the liver capsule. The feedback may be used to prompt a sonographer to improve parallelism between the liver capsule and the transducer array. The design of layered estimated models which improve the accuracy of sound speed estimation may benefit from detecting liver capsules when the depth and angle are fed back in real-time in this manner. Liver B-mode image quality may also be improved using corrected sound speeds from more accurate speed-of-sound estimation accounting for liver capsule segmentation.

Although liver capsule depth and angle detection has been described with reference to several exemplary embodiments, it is understood that the words that have been used are words of description and illustration, rather than words of limitation. Changes may be made within the purview of the appended claims, as presently stated and as amended, without departing from the scope and spirit of liver capsule depth and angle detection in its aspects. Although liver capsule depth and angle detection has been described with reference to particular means, materials and embodiments, liver capsule depth and angle detection is not intended to be limited to the particulars disclosed; rather liver capsule depth and angle detection extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. An ultrasound system comprising:
a memory that stores instructions; and
a processor that executes the instructions, wherein, when executed by the processor the instructions cause the ultrasound system to:
obtain ultrasound images from a transducer array of an ultrasound probe;
detect, based on the ultrasound images, a depth of a liver capsule relative to the transducer array and an angle of the transducer array relative to the liver capsule; and
generate feedback based on the depth and the angle.

2. The ultrasound system of claim 1, further comprising:
the ultrasound probe including the transducer array; and
a display (180) configured to display the feedback generated based on the depth and angle.

3. The ultrasound system of claim 2, wherein, when executed by the processor, the instructions further cause the ultrasound system to:
display the liver capsule in ultrasound images on the display; and
mark the liver capsule in the ultrasound images displayed on the display.

4. The ultrasound system of claim 1, wherein, when executed by the processor, the instructions further cause the ultrasound system to:
filter image data of the ultrasound images to remove speckle variability;
generate first derivatives of the filtered image data to identify changes in signal intensities relative to depth;
apply a threshold to the first derivatives of the filtered image data to identify possible liver capsule boundaries;
for each image line, identify a last point which passes the threshold inside a search window as representing the liver capsule;
eliminate outliers of possible capsule boundary points; and
draw the liver capsule using linear fitting.

5. The ultrasound system of claim 2, wherein, when executed by the processor, the instructions further cause the ultrasound system to:
determine whether a user interface has been activated; and
detect the depth and the angle based on determining that the user interface has been activated.

6. The ultrasound system of claim 2, wherein, when executed by the processor, the instructions further cause the ultrasound system to:
compare the angle to a threshold, wherein the feedback comprises the depth, the angle, and a warning when the angle is greater than the threshold.

7. The ultrasound system of claim 2, wherein, when executed by the processor, the instructions further cause the ultrasound system to:
adjust a threshold used to detect the liver capsule based on user input.

8. The ultrasound system of claim 2, wherein, when executed by the processor, the instructions further cause the ultrasound system to:
measure a thickness of the liver capsule; and
compare a measurement of the thickness of the liver capsule to a threshold.

9. The ultrasound system of claim 1, wherein the memory and the processor are implemented in an ultrasound base.

10. An ultrasound probe comprising:
a transducer array;
a memory that stores instructions; and
a processor that executes the instructions, wherein, when executed by the processor, the instructions cause the ultrasound probe to:
obtain ultrasound images from the transducer array;
detect, based on the ultrasound images, a depth of a liver capsule relative to the transducer array and an angle of the transducer array relative to the liver capsule; and
generate feedback based on the depth and the angle.

11. The ultrasound probe of claim 10, wherein, when executed by the processor, the instructions cause the ultrasound probe to:
output the feedback for display on a display.

12. The ultrasound probe of claim 11, wherein, when executed by the processor, the instructions further cause the ultrasound probe to:
accept input from a user interface; and mark the liver capsule in the ultrasound images for display on the display.

13. The ultrasound probe of claim 10, wherein, when executed by the processor, the instructions further cause the ultrasound probe to:
filter image data of the ultrasound images to remove speckle variability;
generate first derivatives of the filtered image data to identify changes in signal intensities relative to depth;
apply a threshold to the first derivatives of the filtered image data to identify possible liver capsule boundaries;
for each image line, identify a last point which passes the threshold inside a search window as representing the liver capsule;
eliminate outliers of possible capsule boundary points; and
draw the liver capsule using linear fitting.

14. The ultrasound probe of claim 11, wherein, when executed by the processor, the instructions further cause the ultrasound probe to:
determine whether a user interface has been activated; and
detect the depth and the angle based on determining that the user interface has been activated.

15. The ultrasound probe of claim 11, wherein, when executed by the processor, the instructions further cause the ultrasound probe to:
compare the angle to a threshold, wherein the feedback comprises the depth, the angle, and a warning when the angle is greater than the threshold.

16. The ultrasound probe of claim 11, wherein, when executed by the processor, the instructions further cause the ultrasound probe to:
adjust a threshold used to detect the liver capsule based on user input.

17. The ultrasound probe of claim 11, wherein, when executed by the processor, the instructions further cause the ultrasound probe to:
measure a thickness of the liver capsule; and
compare a measurement of the thickness of the liver capsule to a threshold.

18. A method of operating an ultrasound probe comprising a transducer array, a memory that stores instructions, and a processor that executes the instructions, the method comprising:
when the instructions are executed by the processor, obtaining ultrasound images from the transducer array;
detecting, based on the ultrasound images, a depth of a liver capsule relative to the transducer array and an angle of the transducer array relative to the liver capsule; and
generating feedback based on the depth and the angle.
